(19) United States
(12) Patent Application Publication
(10) Pub. No.: US 2023/0384328 A1
(43) Pub. Date: Nov. 30, 2023

(54) Creatinine Assay Kit with Desirable Stability and Capable of Rapidly Eliminating Drug Interference

(71) Applicant: Zhongshan BGH Biochem Co., Ltd., Zhongshan City (CN)

(72) Inventor: Yongbiao Yu, Zhongshan City (CN)

(73) Assignee: Zhongshan BGH Biochem Co., Ltd., Zhongshan City (CN)

(21) Appl. No.: 17/751,741

(22) Filed: May 24, 2022

(51) Int. Cl. G01N 33/70

(52) U.S. Cl. CPC G01N 33/70

(57) ABSTRACT

Provided is a creatinine assay kit with desirable stability and capable of rapidly eliminating drug interference. The creatinine assay kits include a first reagent R1 and a second reagent R2. The first reagent R1 comprises a first buffer solution, creatinase, sarcosine oxidase, ascorbic acid oxidase, peroxidase or catalase, serum albumin, a first non-ionic surfactant, a first preservative, Trinder's reactant A, laccase, a metal salt, or a combination thereof. The second reagent R2 comprises a second buffer solution, serum albumin, creatininase, a second preservative, Trinder's reactant B, or a combination thereof. The creatinine assay kits eliminate or significantly reduce inaccurate creatinine determination due to negative deviation caused by serum samples containing calcium dobesilate and/or etamsylate.

### FIELD OF THE INVENTION

The present disclosure relates to medical testing, and in particular relates to a creatinine assay kit with a desirable stability and capable of rapidly eliminating drug interference.

### BACKGROUND OF THE INVENTION

Creatinine is one of the main indicators that is often tested for clinical examination of renal function. There are two main methods for determining a serum level of creatinine. One is the kinetic alkaline picrate method (“Jaffe's method”) and the other is sarcosine oxidase method. Due to narrow linear range, poor specificity, and detection deviations caused by easy interference of other substances in samples (such as ketone bodies, ascorbic acid, bilirubin, cephalosporin, and dopamine), the Jaffe's method has been gradually withdrawn from the market; thereby, leaving the sarcosine oxidase method as the most commonly used test method for determining serum creatinine level. A schematic illustration of detection of serum creatinine using the sarcosine oxidase method is shown below:

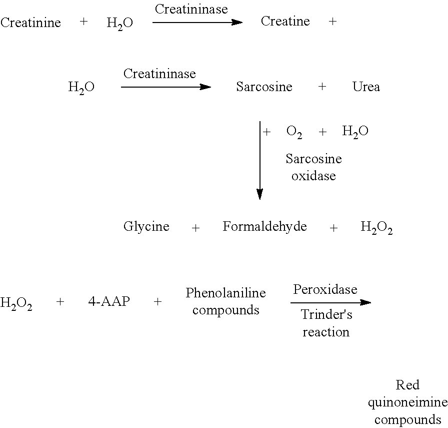 [SMILES: O ; O ; "O2" ; O ; "H2O2" ; "H2O2"]

The current commercially available assay kits for sarcosine oxidase method each include dual reagents. A first reagent R1 includes creatinase, and sarcosine oxidase decomposes creatine in serum to generate the H₂O₂, and H₂O₂ reacts with the peroxidase and a Trinder's reagent to form a colorless compound, or H₂O₂ produced by serum creatine is eliminated using catalase. The resulting H₂O₂ is decomposed into water and oxygen, and then a second reagent R2 is added. The second reagent R2 includes creatininase, another Trinder's reagent, and sodium azide. Creatininase hydrolyzes the creatinine into creatine, and level of creatinine in serum is determined using creatinase, sarcosine oxidase, peroxidase, 4-AAP of the Trinder's reagent, and phenolic compounds of another Trinder's reagent in the first reagent R1. If included in the first reagent R1, catalase is inactivated by sodium azide in the second reagent R2. The generated H₂O₂ as a strong oxidant is consumed by reducing agent in the serum to cause negative interferences.

The creatinine is the most important indicator of renal function. Calcium dobesilate-based drugs, commonly used in treatment and protection of microvascular lesions caused by kidney diseases, have strong reducing properties and can directly react with H₂O₂ to consume H₂O₂. In addition, etamsylate-based drugs, commonly used in prevention and treatment of excessive bleeding in surgical operations, also have strong reducing properties. Both of these two drugs will consume H₂O₂ produced by creatininase method commonly used in hospitals, resulting in serious negative deviations in a creatinine value, and further leading to misjudgment by doctors during diagnosis and treatment.

The negative interference of calcium dobesilate and etamsylate on creatinine detection by the sarcosine oxidase method causes serious problems and great inconvenience to patients who are administered these drugs. For example, patients with diabetes are generally accompanied by renal insufficiency; the negative interference of the administered calcium dobesilate on the creatinine detection by sarcosine oxidase method may cause incorrect assessment of the renal function in diabetic patients, masking the disease and delaying the diagnosis. For surgical patients, the negative interference of etamsylate on the creatinine detection by sarcosine oxidase method may make it impossible to correctly assess the renal function, causing postoperative complications to affect postoperative recovery of the patients.

Therefore, there is a need for a more accurate serum creatinine assay kit to avoid these potentially life-threatening complications or incorrect serum creatinine determinations.

### SUMMARY OF THE INVENTION

The present disclosure provides a creatinine assay kit that significantly reduces or even eliminates negative deviation interference of creatinine determination caused by calcium dobesilate-based and etamsylate-based drugs in serum. In particular, the present disclosure avoids inaccurate creatinine determination due to negative deviation caused by serum samples containing calcium dobesilate and etamsylate when the creatinine is determined by clinical tests in hospitals, thereby avoiding misjudgment by physicians during the treatment. The assay kit has desirable stability, simple and rapid operations, and accurate determination, and is suitable for serum creatinine detection by an automatic biochemical analyzer.

The present disclosure further discloses a method for preparing of a creatinine assay kit capable of eliminating or significantly reducing negative deviation interference of creatinine determination caused by calcium dobesilate-based and etamsylate-based drugs in serum.

The present disclosure also provides a creatinine assay kit capable of eliminating or significantly reducing negative deviation interference of creatinine determination caused by calcium dobesilate-based and etamsylate-based drugs in serum, including a first reagent R1 and a second reagent R2.

The first reagent R1 is selected from the group consisting of a first buffer solution, creatinase, sarcosine oxidase, ascorbic acid oxidase, peroxidase or catalase, serum albumin, a non-ionic surfactant, a first preservative, Trinder's reactant A, laccase, and a metal salt.

The second reagent R2 is selected from the group consisting of a second buffer solution, serum albumin, creatininase, a second preservative, an optional non-ionic surfactant, and Trinder's reactant B. Laccase can oxidize calcium dobesilate and etamsylate into benzoquinone compounds using oxygen (02); the metal salts or vanadium pentoxide or vanadium oxytrichloride can be used as an oxidant to oxidize calcium dobesilate and etamsylate, thus avoiding consuming H₂O₂ generated during the creatinine determination. Among the first reagent R1, laccase or oxidized metal salts or vanadium pentoxide or vanadium oxytrichloride components can also be stripped or presented separately from the first reagent R1, and then mixed to form the first reagent R1 before being used together alone.

In some embodiments, ascorbic acid oxidase is optionally included in the first reagent R1. Typically, the amount of ascorbic acid oxidase present in the first reagent R1 ranges from about 5 KU/L to about 10 KU/L, often from about 5.5 KU/L to about 10 KU/L. Ascorbic acid oxidase within the above range can effectively reduce interference caused by ascorbic acid in the creatinine determination. In some examples, the amount of ascorbic acid oxidase present in the first reagent R1 can be 5.5 KU/L, 6.4 KU/L, 7.5 KU/L, 8.8 KU/L, 8.6 KU/L, 6 KU/L, 7.2 KU/L, 9.2 KU/L, 9.5 KU/L, or 10 KU/L.

In one particular embodiment, laccase is laccase derived from Aspergillus sp. which is available from Sigma-Aldrich with catalog number SAE0050 laccase. Laccase is a copper-containing and polyphenol-based oxidoreductase, and can oxidize various phenolic compounds using oxygen molecules in water to generate quinone compounds. In the process, oxygen molecules are reduced to water. Therefore, laccase is an environmental-friendly enzyme commonly used in sewage treatment to eliminate phenolic compounds in the sewage. Calcium dobesilate and etamsylate are both reducing agents containing a phenol ring structure, which can be oxidized and eliminated by laccase; however, laccase generally has the highest activity and stability at pH value of about pH 2 to about pH 4. However, reagents of the creatininase assay kit typically operates at a pH value ranging from about pH 7 to about pH 9.

Surprisingly and unexpectedly, the present inventors have discovered that laccase derived from Aspergillus sp. (e.g., SAE0050 laccase from Sigma-Aldrich) was particularly useful in creatinine assay kit of the disclosure. Laccase has desirable activity and stability at the pH value range of about pH 7.0 to about pH 9.0, and can be added to the first reagent R1 of creatininase assay kit to eliminate interference of calcium dobesilate and etamsylate.

Still in other embodiments, the metal salt is selected from the group consisting of oxidized nickelate, ferrite, cobaltate, chromate, manganate, and vanadate. In some instances, vanadate is one or more selected from the group consisting of polyvanadate, metavanadate, vanadium pentoxide, and vanadium oxytrichloride. In other embodiments of the present disclosure, acid salts of metal elements at positions 23, 24, 25, 26, 27, and 28 in the periodic table of elements are used such as, but not limited to, nickelate, cobaltate, chromate, manganate, and vanadate in oxidation state with a high redox potential. Useful vanadates include, but are not limited to, the metavanadate, polyvanadate, and vanadium pentoxide. In addition, vanadium oxytrichloride can also be used as an oxidant to eliminate the negative deviations caused by the interference of calcium dobesilate and etamsylate in serum during the creatinine determination, without affecting the stability of each component in the first reagent R1 and the second reagent R2.

Yet in other embodiments, from about 5 mM to about 200 mM of the first buffer salt solution concentration may be used in the first reagent R1. In other embodiments, the pH of the first buffer solution can range from about pH 7.0 to about pH 9.0. Still in other embodiments, from about 5 ku/L to about 50 ku/L of creatinase is present in the first reagent R1. In further embodiments, from about 5 ku/L to about 50 ku/L of sarcosine oxidase is present in the first reagent R1. Yet in further embodiments, from about 1 ku/L to about 10 ku/L of ascorbic acid oxidase is present in the first reagent R1. Still in further embodiments, from about 1 ku/L to about 10 ku/L of the peroxidase or from about 100 ku/L to about 500 ku/L of the catalase, from about 1 g/L to about 2 g/L of serum albumin, and from about 0.5 mM to about 5 mM/L of the Trinder's reactant A is used in the first reagent R1. In some cases, the Trinder's reactant A is 4-aminoantipyrine (4-AAP). The amount of preservative in the first reagent R1 can be from about 0.005% by weight to about 0.2% by weight. If laccase is used to eliminate the negative interference of calcium dobesilate and etamsylate, then the amount of laccase in the first reagent R1 is from about 0.1 ku/L to about 10 ku/L. If one of the metal acid salt oxidant or the vanadium pentoxide or vanadium oxytrichloride oxidant or a mixture of two or more thereof is used, then the oxidant has a concentration of from about 0.01 mM to about 10 mM. The metal salt with high redox potential can be sodium salts, potassium salts, ammonium salts of a metal acid.

Still in other embodiments, the first buffer solution is selected from one or more of BES, BICINE, DIPSO, EPPS, PIPES, HEPES, MOPS, TAPS, TAPSO, TES, Tricine, Tris, Gly-Gly, and phosphate.

Yet in other embodiments, when the second buffer solution is present in the reagent R2, the second buffer solution is selected from one or more of BES, BICINE, DIPSO, EPPS, PIPES, HEPES, MOPS, TAPS, TAPSO, TES, TRICINE, TRTS, Gly-Gly, and phosphate.

In further embodiments, the first reagent R1 and the second reagent R2 each may independently include a surfactant at an amount of from about 0.01% by weight to about 0.3% by weight. Typically, the surfactant is a non-ionic surfactant. In some instances, the surfactant is selected from the group consisting of TRITON, TWEEN, BRIJ, EMULGEN, and a combination thereof.

Still in other embodiments, the first and the second preservatives are independently selected from the group consisting of Proclin, gentamicin sulfate, PARABEN, chloramphenicol, cycliheximide, and sodium azide.

Yet in other embodiments, the amount of second buffer solution in the second reagent R2 ranges from about 10 mM to about 200 mM. In some instances, the pH of the second buffer solution ranges from about pH 6.5 to about pH 9.0. Still in other instances, the amount of Trinder's reactant B in the second reagent R2 ranges from about 0.1 mM to about 20 mM. Yet in other instances, the amount of creatininase in the second reagent R2 ranges from about 50 ku/L to about 1,000 ku/L.

In further embodiments, the Trinder's reactant B is selected from one or more of TOOS, DHBS, DAOS, HDAOS, TBHBA, TOPS, TODB, and 4-chlorophenol. These components are mixed with various phenol or aniline derivatives of Trinder's reactants to generate chromogen components. Typically, Trinder's reactant A is contained in the first reagent R1 and Trinder's reactant B is contained in the second reagent R2.

For the first reagent R1 and the second reagent R2, the first reagent R1 includes laccase that eliminates the interference of calcium dobesilate and etamsylate. It is possible to oxidize calcium dobesilate and etamsylate in serum before adding creatininase (in the second reagent R2) to avoid the consumption of H₂O₂ generated during the creatinine determination; and sodium azide is added to the reagent R2, and the reagent R2 is added after a reaction of the reagent R1 with components in the serum is completed to inactivate laccase and catalase. This avoids the effect of addition of creatininase on the determination of creatinine.

The present disclosure further discloses a method for preparing the creatinine assay kit capable of eliminating negative deviation interference of creatinine determination caused by calcium dobesilate-based and etamsylate-based drugs in serum, including the following steps:

Preparation of the first reagent R1: To the first buffer solution was added a preservative and laccase or the metal salt. The mixture was stirred for 1 h, and pH was adjusted to pH 8.0 with 1N hydrochloric acid or sodium (or potassium) hydroxide. To the resulting mixture was added Trinder's reactant A, a non-ionic surfactant, and BSA at a weight percent of 0.1%. After stirring the mixture for 1 h, the solution was allowed to stand at 2° C. to 8° C. overnight, after which the following were added: creatinase, sarcosine oxidase, peroxidase or catalase, and ascorbic acid oxidase. The mixture was then stirred for 1 h to obtain first reagent R1. Typically, the metal salt is one or more selected from the group consisting of the nickelate, ferrite, cobaltate, chromate, manganate, and vanadate. Often Trinder's reactant A is the 4-aminoantipyrine.

Preparation of the second reagent R2: To the second buffer solution, whose pH was adjusted to pH 8.0 with 1N hydrochloric acid or sodium (or potassium) hydroxide, was added Trinder's reactant B, a second preservative, a non-ionic surfactant and serum albumin. The resulting mixture was stirred for 1 h and allowed to cool. The mixture was held at 2° C. to 8° C. overnight, after which creatininase was added to obtain second reagent R2.

The assay kit includes the first reagent R1 and the second reagent R2. The first reagent R1 is mixed with a specimen, such that creatine in the specimen is eliminated. For example, the therapeutic drugs calcium dobesilate or etamsylate contained in the specimen can be oxidized by laccase or the metal salts or vanadium pentoxide or vanadium oxytrichloride in the first reagent R1. This avoids or prevents H₂O₂ produced by creatininase from being consumed by the drugs calcium dobesilate or etamsylate. The second reagent R2 includes the Trinder's reactant B, a phenolic compounds, that reacts with H₂O₂ produced by a series of reactions between 4-AAP and creatininase in the first reagent R1. Reaction of Trinder's reactant B with hydrogen peroxide results in a color reaction, whose absorbance is determined to calculate the level of creatinine.

One of the beneficial effects of creatinine assay kits of the disclosure include, but not limited to, eliminating negative deviation interference of creatinine determination caused by calcium dobesilate-based and etamsylate-based drugs in serum. Currently, conventional sarcosine oxidase-based methods on the market involve the use of dual reagents and a Trinder's reaction to determine the level of creatinine in serum. The first reagent R1 includes phenol or aniline compounds commonly used in the Trinder's reactant, such as the 4-chlorophenol, TOOS, DHBS, DAOS, HDAOS, TBHBA, TOPS, and TODB; and the second reagent R2 includes 4-AAP. In a series of experiments, it was found by the present inventor that 4-AAP must be added to the first reagent R1 to prevent inactivation or destruction of laccase or the above metal salts or vanadium pentoxide and vanadium oxytrichloride and other components in the first reagent R1. Another reagent, phenolic compounds, also is present in creatinine assay kit of the disclosure along with creatininase in the second reagent R2 to avoid interference from calcium dobesilate and etamsylate. The creatinine assay kit of the disclosure is stable at 2° C. to 8° C. for one year or longer.

The creatinine assay kit of the disclosure eliminates or significantly reduces negative deviation interference caused by the presence of calcium dobesilate-based and/or etamsylate-based drugs in serum when determining the level of creatinine. Without being bound by any theory, it is believed that creatine produced from a serum sample by the actions of the first reagent R1 (e.g., via actions of creatinase and sarcosine oxidase) is decomposed by catalase or peroxidase and 4-AAP present in the first reagent R1. This decomposition of creatine using the first reagent R1 eliminates inaccurate reading caused by interference from calcium dobesilate-based and/or etamsylate-based drugs in serum. The first reagent R1 also includes ascorbic acid oxidase, which can eliminate ascorbic acid in the serum to avoid the interference caused by the ascorbic acid.

The creatinine assay kit of the disclosure provides desirable stability, simple and rapid operations, eliminating the negative interference of calcium dobesilate and etamsylate with high accuracy, thereby significantly reducing or eliminating possible misjudgment by physicians in clinical diagnosis, and enabling precise medication and treatment. The assay kit is suitable for use on an automatic biochemical analyzer. Dual reagents can be loaded on an appropriate diagnostic machine, providing simple operations, safety and reliability.

The present disclosure further provides a method for preparing the creatinine assay kit capable of eliminating or significantly reducing negative deviation interference of creatinine determination caused by calcium dobesilate-based and etamsylate-based drugs in serum. The method is unique, features a simple process, and is suitable for batch preparation.

### BRIEF DESCRIPTION OF THE DRAWINGS

FIG. 1 shows a schematic diagram of absorbance test.

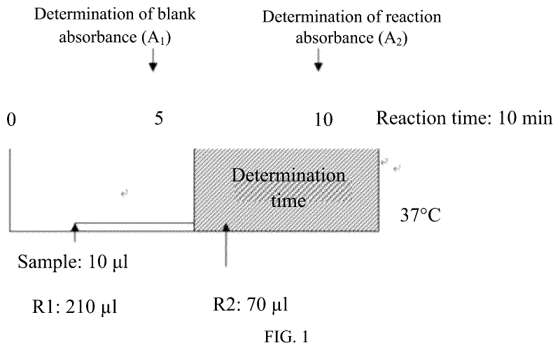

### DETAILED DESCRIPTION OF THE INVENTION

The present disclosure will now be described with regard to the accompanying drawings and the experimental procedures and results discussed below, which assist in illustrating various features of the invention. In this regard, the present disclosure generally relates to a creatinine assay kit that eliminates or significantly reduces negative deviation interference of creatinine level determination caused by the presence of calcium dobesilate-based and/or etamsylate-based drugs in serum.

The interference of calcium dobesilate and etamsylate was determined by currently commercially-available creatininase kits approved by the China National Medical Products Administration. The results are shown in Table 1.

Components of conventional commercially available reagents were as described in user manuals. Briefly, the main component of conventional creatinine assay kit included a reagent R₁, a reagent R₂ and a calibrator. The reagent R₁ included: a tris(hydroxymethyl)aminomethane (Tris) buffer, creatine amidinohydrolase, sarcosine oxidase, ascorbic acid oxidase, sodium 2-hydroxy-3-m-toluidine propanesulfonate (TOOS), catalase, and a surfactant. The reagent R₂ included: Tris buffer, creatinine aminohydrolase, 4-aminoantipyrine, and peroxidase. The calibrator was an aqueous solution containing creatinine, with concentration marked on label, which is traceable to a Randox CAL3 calibrator.

Test methods: The dual reagents of conventional creatinine assay kit were used directly without preparation. Test conditions were as follows: sample (S): 10 μl, reagent 1 (R₁): 210 μl, reagent 2 (R₂): 70 μl, temperature: 37° C. Test type: endpoint method, primary wavelength: 546 nm, secondary wavelength: 660 nm, reaction direction: INCREASE. The method included following procedures: the sample was mixed with the reagent R₁, the reagent R₂ was added after 5 min at 37° C., and an absorbance of the reaction was determined 5 min after adding R₂.

Calibration procedure: Calibration was conducted using the calibrator every time when a reagent batch was changed. After calibration, each laboratory conducted verification with quality control materials. Recalibration was required if quality control results were not within acceptable ranges.

Determination instruments and parameters:
Unit: μmol/L, normal value≤70 μmol/L;
Determination instrument: Hitachi 7180;
Determination parameters: R: R2: S (sample volume)=210: 70: 10 μl;
Primary/secondary wavelength: 546 nm/660 nm;
2POINT END, INC.

TABLE 1
Determination interference results obtained using existing (conventional
commercially available) dual-reagent creatininase assay kit

| Control | 1 | 2 | 3 | Average value | Relative deviation |
|---|---|---|---|---|---|
| Mother liquor | 139 | 139 | 139 | 139 | |
| Calcium dobesilate 8 mg/L | 121 | 121 | 121 | 121.00 | −12.9% |
| Calcium dobesilate 16 mg/L | 103 | 102 | 102 | 102.3 | −26.4% |
| Calcium dobesilate 32 mg/L | 82 | 81 | 82 | 81.7 | −41.2% |
| Calcium dobesilate 64 mg/L | 45 | 46 | 45 | 45.3 | −67.4% |
| Etamsylate 12.5 mg/L | 114 | 114 | 113 | 113.70 | −18.2% |
| Etamsylate 25 mg/L | 90 | 91 | 91 | 90.3 | −35.0% |
| Etamsylate 50 mg/L | 60 | 60 | 60 | 60.00 | −56.8% |
| Etamsylate 100 mg/L | 46 | 46 | 45 | 45.70 | −67.1% |

Conclusion: The determination results clearly show that both calcium dobesilate and etamsylate cause significantly negative deviations in the amount of creatinine present in the sample.

The creatinine assay kit capable of eliminating negative deviation interference of creatinine determination caused by calcium dobesilate-based and etamsylate-based drugs in serum of the present disclosure will be described below in conjunction with Examples 1-4.

TABLE 2
Reagent components of Examples 1-4

| | | Example 1 | Example 2 | Example 3 | Example 4 |
|---|---|---|---|---|---|
| R1 | Buffer solution | TRIS buffer | EPPS buffer | TRIS buffer | HEPES buffer |
| | Creatinase | Creatinase | Creatinase | Creatinase | Creatinase |
| | Sarcosine | Sarcosine | Sarcosine | Sarcosine | Sarcosine |
| | oxidase | oxidase | oxidase | oxidase | oxidase |
| | Peroxidase/ | Peroxidase | Peroxidase | Catalase | Peroxidase |
| | catalase | | | | |
| | Laccase/ | Laccase | Vanadium | Sodium | Sodium |
| | metal salt | | pentoxide | ferrate | metavanadate |
| | Trinder's reactant | 4-AAP | 4-AAP | 4-AAP | 4-AAP |
| | A | | | | |
| | Serum albumin | Bovine serum | Bovine serum | Bovine serum | Bovine serum |
| | | albumin | albumin | albumin | albumin |
| | Non-ionic | TRITON | TRITON | TRITON | TRITON |
| | surfactant | X-100 | X-100 | X-100 | X-100 |
| | Preservative | PARABEN | Chloramphenicol | chloramphenicol | paraben |
| | | preservative | | | |
| | Ascorbic acid | Ascorbic acid | Ascorbic acid | Ascorbic acid | Ascorbic acid |
| | oxidase | oxidase | oxidase | oxidase | oxidase |
| R2 | Buffer solution | TRIS buffer | EPPS buffer | TRIS buffer | HEPES buffer |
| | Creatininase | Creatininase | Creatininase | Creatininase | Creatininase |
| | Serum albumin | Bovine serum | Bovine serum | Bovine serum | Bovine serum |
| | | albumin | albumin | albumin | albumin |
| | Trinder's reactant | TOOS | TOOS | TOOS | DHBS |
| | B | | | | |
| | Preservative | Sodium azide | Sodium azide/ | Chloramphenicol | Proclin |
| | | | chloramphenicol | | |
| | Non-ionic | TRITON | TRITON | TRITON | TRITON |
| | surfactant | X-100 | X -100 | X -100 | X-100 |

The specific components of the creatinine reagent according to the present disclosure were described as follows (by weight percentage in the examples):

Reagent R1 components:

| TRIS buffer | 50 mM at pH 8.0; | |
|---|---|---|
| Creatinase | 25 | ku/L; |
| Sarcosine oxidase | 15 | ku/L; |
| Peroxidase | 10 | ku/L; |
| Ascorbic acid oxidase | 10 | ku/L; |
| Laccase | 1 | ku/L; |
| 4-AAP | 3 | mM; |

| | |
|---|---|
| Bovine serum albumin | 0.1%; |
| TRITON X-100 | 0.2%; |
| PARABEN preservative | 0.1%; |

Reagent R2 components:

| TRIS buffer | 50 mM at pH 8.0; | |
|---|---|---|
| Creatininase | 300 | ku/L; |
| Bovine serum albumin | 0.1%; | |
| TOOS | 10 | mM; |

| | |
|---|---|
| Sodium azide | 0.1%; |
| TRITON X-100 | 0.1%. |

### Example 2

Reagent R1 components:

| EPPS buffer | 50 mM at pH 8.0; | |
|---|---|---|
| Creatinase | 25 | ku/L; |
| Sarcosine oxidase | 15 | ku/L; |
| Peroxidase | 5.5 | ku/L; |
| Ascorbic acid oxidase | 6.4 | ku/L; |
| Vanadium pentoxide | 1 | mM; |
| 4-AAP | 3 | mM; |

| | |
|---|---|
| Bovine serum albumin | 0.1%; |
| TRITON X-100 | 0.2%; |
| Chloramphenicol | 0.1%; |

Reagent R2 components:

| EPPS buffer | 50 mM at pH 8.0; | |
|---|---|---|
| Creatininase | 500 | ku/L; |
| Bovine serum albumin | 0.1%; | |
| TOOS | 10 | mM; |

| | |
|---|---|
| Sodium azide/chloramphenicol | 0.1%; |
| TRITON X-100 | 0.1%. |

### Example 3

Reagent R1 components:

| TRIS buffer | 50 mM at pH 8.0; | |
|---|---|---|
| Creatinase | 25 | ku/L; |
| Sarcosine oxidase | 15 | ku/L; |
| Catalase | 100 | ku/L; |
| Ascorbic acid oxidase | 7.5 | ku/L; |
| Sodium ferrate | 2 | mM; |
| 4-AAP | 3 | mM; |

| | |
|---|---|
| Bovine serum albumin | 0.1%; |
| TRITON X-100 | 0.2%; |
| chloramphenicol | 0.1%; |

Reagent R2 components:

| TRIS buffer | 50 mM at pH 8.0; | |
|---|---|---|
| Creatininase | 500 | ku/L; |
| Bovine serum albumin | 0.1%; | |
| TOOS | 10 | mM; |

| | |
|---|---|
| Chloramphenicol/sodium azide | 0.1%; |
| TRITON X-100 | 0.1%. |

### Example 4

Reagent R1 components:

| HEPES buffer | 50 mM at pH 8.0; | |
|---|---|---|
| Creatinase | 25 | ku/L; |
| Sarcosine oxidase | 15 | ku/L; |
| Peroxidase | 10 | ku/L; |
| Ascorbic acid oxidase | 6 | ku/L; |
| Sodium metavanadate | 5 | mM; |
| 4-AAP | 3 | mM; |

| | |
|---|---|
| Bovine serum albumin | 0.1%; |
| TRITON X-100 | 0.2%; |
| Sodium azide/PARABEN | 0.1%; |

Reagent R2 components:

| HEPES buffer | 50 mM at pH 8.0; | |
|---|---|---|
| Creatininase | 500 | ku/L; |
| Bovine serum albumin | 0.1%; | |
| DHBS | 10 | mM; |

| | |
|---|---|
| Proclin | 0.1%; |
| TRITON X-100 | 0.1%. |

### Preparation Methods of Examples 1-4

Preparation of the first reagent R1: To the first buffer solution was added the preservative, and laccase or metal salt. The mixture was stirred for 1 h and the pH was adjusted to 8.0 using 1N hydrochloric acid or sodium (or potassium) hydroxide. To the resulting solution was then added the Trinder's reactant A, the non-ionic surfactant, and 0.1% by weight of BSA and stirred for 1 h. The mixture was then allowed to stand at 2° C. to 8° C. overnight, after which creatinase, sarcosine oxidase, peroxidase or catalase, and ascorbic acid oxidase were added and stirred for 1 h.

Preparation of the second reagent R2: To the second buffer solution at pH 8.0 (adjusted with 1N hydrochloric acid or the sodium (or potassium) hydroxide) was added Trinder's reactant B, preservative, the non-ionic surfactant, and serum albumin. The resulting mixture was stirred for 1 h, cooled, and allowed to stand at 2° C. to 8° C. overnight. To the resulting mixture was added creatininase to obtain the second reagent R2.

Test Methods

The first and the second reagents R1 and R2, respectively, obtained in Examples 1-4 were placed in corresponding reagent positions of the automatic coagulation analyzer (Hitachi 7180); 210 μl of first reagent R1, 70 μl of second reagent R2, and 10 μl of calibration solution or a specimen were aspirated. The calibration solution had a concentration of 300 μM, primary and secondary analyzer wavelengths were 546 nm and 660 nm, respectively. A two-point endpoint method was adopted with a reaction direction of INC (INCREASE); the creatinine value of each specimen containing different concentrations of calcium dobesilate or etamsylate was determined by the calibration solution.

The corresponding results of using the first and the second reagents R1 and R2, respectively, in Examples 1-4 are shown in Tables 3-6, respectively.

TABLE 3
Determination results of Example 1 (unit: μmol/L)

| Determined value | 1 | 2 | 3 | Average value | Relative deviation |
|---|---|---|---|---|---|
| Mother liquor | 139 | 140 | 139 | 139.30 | |
| Calcium dobesilate 8 ml/L | 141 | 137 | 138 | 138.70 | −0.95% |
| Calcium dobesilate 16 ml/L | 141 | 141 | 140 | 140.7 | 1.0% |
| Calcium dobesilate 32 ml/L | 141 | 141 | 139 | 140.30 | 0.13% |
| Calcium dobesilate 64 ml/L | 139 | 138 | 138 | 138.3 | −0.017% |
| Etamsylate 12.5 mg/L | 140 | 137 | 139 | 138.70 | −0.15% |
| Etamsylate 25 mg/L | 141 | 140 | 139 | 140.00 | 0.15% |
| Etamsylate 50 mg/L | 139 | 140 | 138 | 144.00 | −0.2% |
| Etamsylate 100 mg/L | 139 | 138 | 137 | 138 | −1.0% |

TABLE 4
Determination results of Example 2 (unit: μmol/L)

| Determined value | 1 | 2 | 3 | Average value | Relative deviation |
|---|---|---|---|---|---|
| Mother liquor | 148 | 146 | 147 | 147.01 | |
| Calcium dobesilate 8 ml/L | 149 | 148 | 147 | 148.01 | 0.7% |
| Calcium dobesilate 16 ml/L | 149 | 147 | 148 | 148.01 | 0.7% |
| Calcium dobesilate 32 ml/L | 148 | 148 | 149 | 151.01 | 1.0% |
| Calcium dobesilate 64 ml/L | 147 | 148 | 148 | 152.01 | 0.5% |
| Etamsylate 12.5 mg/L | 147 | 146 | 147 | 146.70 | −0.2% |
| Etamsylate 25 mg/L | 148 | 147 | 147 | 147.30 | 0.2% |
| Etamsylate 50 mg/L | 147 | 148 | 148 | 147.70 | 0.5% |
| Etamsylate 100 mg/L | 147 | 148 | 146 | 147.0 | 0.0% |

TABLE 5
Determination results of Example 3 (unit: μmol/L)

| Determined value | 1 | 2 | 3 | Average value | Relative deviation |
|---|---|---|---|---|---|
| Mother liquor | 152 | 152 | 152 | 152.01 | |
| Calcium dobesilate 8 ml/L | 154 | 153 | 152 | 153.01 | 0.7% |
| Calcium dobesilate 16 ml/L | 152 | 152 | 152 | 152.01 | 0.0% |
| Calcium dobesilate 32 ml/L | 152 | 152 | 151 | 151.7 | −0.2% |
| Calcium dobesilate 64 ml/L | 152 | 151 | 151 | 151.3 | −0.5% |
| Etamsylate 12.5 mg/L | 153 | 153 | 153 | 153.01 | 0.7% |
| Etamsylate 25 mg/L | 155 | 153 | 153 | 153.7 | 1.1% |
| Etamsylate 50 mg/L | 155 | 153 | 153 | 153.7 | 1.1% |
| Etamsylate 100 mg/L | 153 | 154 | 153 | 153.3 | 0.9% |

TABLE 6
Determination results of Example 4 (unit: μmol/L)

| Determined value | 1 | 2 | 3 | Average value | Relative deviation |
|---|---|---|---|---|---|
| Mother liquor | 116 | 115 | 116 | 115.7 | |
| Calcium dobesilate 8 ml/L | 116 | 116 | 115 | 115.7 | 0 |
| Calcium dobesilate 16 ml/L | 116 | 115 | 115 | 115.3 | −0.3% |
| Calcium dobesilate 32 ml/L | 115 | 115 | 115 | 115.0 | −0.6% |
| Calcium dobesilate 64 ml/L | 115 | 115 | 114 | 114.7 | −0.9% |
| Etamsylate 12.5 mg/L | 115 | 115 | 115 | 115.0 | −0.6% |
| Etamsylate 25 mg/L | 115 | 116 | 116 | 115.30 | −0.3% |
| Etamsylate 50 mg/L | 114 | 114 | 115 | 114.30 | −1.0% |
| Etamsylate 100 mg/L | 114 | 114 | 113 | 113.7 | −1.7% |

The experimental results of the assay kit of Examples 1-4 show that the creatinine assay kit of the disclosure is capable of eliminating or significantly reducing negative deviation interference of creatinine determination caused by calcium dobesilate-based and etamsylate-based drugs in serum.

The creatinine assay kit of the disclosure is typically used by mixing the first reagent R1 with a specimen, which ultimately produces sarcosine from creatinine. If any calcium dobesilate and/or etamsylate is present in the specimen, they are oxidized by laccase or the metal salts or vanadium pentoxide or vanadium oxytrichloride in the first reagent R1. Accordingly, in creatinine assay kits of the disclosure, laccase or the ferrate, polymetavanadate and metavanadate with a high redox potential effectively eliminate the interference of calcium dobesilate and/or etamsylate in the creatinine determination, thereby allowing one to obtain an accurate creatinine level determination. In the creatinine assay kits of the disclosure, the second reagent R2 includes the Trinder's reactant B, a phenolic compound, which is reacted with H₂O₂ produced by a series of reactions between 4-AAP and creatininase in the first reagent R1 to proceed color reaction. By determining the absorbance of the color reaction produced by creatinine assay kits of the disclosure allows one to determine the level of creatinine in the serum sample.

In the presence of catalase or peroxidase in reagent R1 and 4-AAP, creatine in serum samples can be decomposed by H₂O₂ produced by creatinase and sarcosinase oxidase in first reagent R1. Removal or decomposition of creatine eliminates any potential interference caused by the presence of creatine. The assay kits of the disclosure are stable, simple to operate, and provide rapid analysis. More significantly, the assay kits of the disclosure eliminate or significantly reduce the negative interference caused by calcium dobesilate and/or etamsylate and provide highly accurate results, thereby avoiding or significantly reducing potential errors in clinical diagnosis. By providing more accurate analysis of serum creatinine level, the assay kits of the disclosure enable physicians to provide more precise medication and treatment protocols to subjects. The assay kits of the disclosure are suitable for use on an automatic biochemical analyzer. One possible method of using the assay kits of the disclosure include loading the dual reagents on the machine, thereby providing simple operations. By automating the process, assay kits of the disclosure can be used safely and reliably. Laccase or ferrate, polymetavanadate and metavanadate with a high redox potential effectively eliminates the interference of calcium dobesilate and/or etamsylate in the creatinine assay to provide accurate results for creatinine level analysis.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

1. A creatinine assay kit comprising:
a first reagent R1 selected from the group consisting of a first buffer solution, creatinase, sarcosine oxidase, ascorbic acid oxidase, peroxidase, serum albumin, a non-ionic surfactant, a first preservative, 4-aminoantipyrine, laccase from Aspergillus sp, chromate, ferrite, vanadium pentoxide, and a combination thereof,
a second reagent R2 selected from the group consisting of a second buffer solution, serum albumin, creatininase, a second preservative, Trinder's reactant B, and a combination thereof; and
an manual providing an instruction that the first reagent R1 and the second reagent R2 must be used in sequence,
wherein
said Trinder's reactant B is selected from the group consisting of sodium 2-hydroxy-3-m-toluidine propanesulfonate (TOOS), 3,5-dichloro-2-hydroxybenzenesulfonic acid sodium salt (DHBS), N-ethyl-n-(2-hydroxy-3-sulfopropyl)-3,5-dimethoxyaniline sodium salt (DAOS), N-(2-hydroxy-3-sulfopropyl)-3,5-dimethoxyaniline sodium salt (HDAOS), 2,4,6-tribromo-3-hydroxybenzoic acid (TBHBA), N-ethyl-N-sulfopropyl-m-toluidine sodium salt (TOPS), N,N-bis(4-sulfobutyl)-3-methylaniline, disodiumsalt (TODB), 4-chlorophenol, and a combination thereof; and
each of said first preservative and said second preservative is independently selected from the group consisting of Proclin, gentamicin sulfate, PARABEN, chloramphenicol, sodium azide, and a combination thereof.

2. (canceled)

3. The creatinine assay kit according to claim 1, wherein said first reagent R1 comprises:
said first buffer solution having a first buffer salt concentration of from about 5 mM to about 200 mM and a pH of from about pH 7.0 to about pH 9.0;
5 ku/L to 50 ku/L of creatinase;
5 ku/L to 50 ku/L of sarcosine oxidase;
1 ku/L to 10 ku/L of peroxidase;
1 ku/L to 10 ku/L of ascorbic acid oxidase;
1 g/L to 2 g/L of serum albumin; and
0.5 mM to 5 mM of Trinder's reactant A,
and wherein when said first preservative is present in a weight percent of from about 0.005% to about 0.2%, then:
(a) the amount of laccase ranges from about 0.1 ku/L to about 10 ku/L; or
(b) the amount of chromate, ferrite, or vanadium pentoxide ranges from about 0.01 mM to about 10 mM.

4. The creatinine assay kit according to claim 1, wherein said first buffer solution is selected from the group consisting of N,N-bis[2-Hydroxyethyl]-2-aminoethanesulfonic acid (BES), N,N-bis[2-Hydroxyethyl]glycine (BICINE), 3-[N,N-bis(2-hydroxyethyl)amino]-2-hydroxy-propanesulfonic acid (DIPSO), N-[2-hydroxyethyl]piperazine-N′-[3-propanesulfonic acid] (EPPS), piperazine-N,N′-bis[2-ethanesulfonic acid] (PIPES), N-[2-hydroxyethyl]piperazine-N′-[2-ethanesulfonic acid](HEPES), 3-[N-morpholino]propanesulfonic acid (MOPS), N-tris[hydroxymethyl]methyl-3-aminopropanesulfonic acid (TAPS), 3-[N-tris(hydroxymethyl)methylamino]-2-hydroxy-propanesulfonic acid (TAPSO), N-tris[hydroxyethyl]methyl-2-aminoethanesulfonic acid (TES), N-tris[hydroxymethyl]methylglycine (Tricine), tris(hydroxymethyl)aminomethane (Tris), glycylglycine (Gly-Gly) a phosphate thereof, and a mixture thereof.

5. The creatinine assay kit according to claim 1, wherein said second buffer solution is selected from the group consisting of BES, BICINE, DIPSO, EPPS, PIPES, HEPES, MOPS, TAPS, TAPSO, TES, TRICINE, TRTS, Gly-Gly, a phosphate thereof, and a mixture thereof.

6. The creatinine assay kit according to claim 1, wherein an amount of said non-ionic surfactant in said first reagent R1 is about 0.01% to 0.3% by weight.

7. The creatinine assay kit according to claim 6, wherein said non-ionic surfactant is selected from the group consisting of TRITON, TWEEN, BRIJ, EMULGEN, and a combination thereof.

8. The creatinine assay kit according to claim 1, wherein said first preservative is selected from the group consisting of Proclin, gentamicin sulfate, PARABEN, chloramphenicol, sodium azide, and a combination thereof.

9. The creatinine assay kit according to claim 1, wherein said second preservative is selected from the group consisting of Proclin, gentamicin sulfate, PARABEN, chloramphenicol, sodium azide, and a combination thereof.

10. The creatinine assay kit according to claim 1, wherein said second buffer solution has a second buffer salt concentration of from about 10 mM to about 200 mM.

11. The creatinine assay kit according to claim 10, wherein a pH of said second buffer solution is from about pH 6.5 to about pH 9.0.

12. The creatinine assay kit according to claim 11, wherein a concentration of Trinder's reactant B in said second reagent R2 is from about 0.1 mM to about 20 mM.

13. The creatinine assay kit according to claim 12, wherein a concentration of creatininase in said second reagent R2 is from about 50 ku/L to about 1,000 ku/L.
